(19) United States
(12) Patent Application Publication
(10) Pub. No.: US 2011/0160554 A1
(43) Pub. Date: Jun. 30, 2011

(54) DEVICE AND METHOD FOR DETERMINING AT LEAST ONE CHARACTERIZING PARAMETER OF MULTILAYER BODY TISSUE

(76) Inventors: Alexander Megej, Zurich (CH); Andreas Caduff, Zurich (CH); Mark Talary, Zurich (CH)

(21) Appl. No.: 12/999,127

(22) PCT Filed: Jun. 18, 2008
(86) PCT No.: PCT/CH08/00275

§ 371 Date: Mar. 10, 2011

(51) Int. Cl. A61B 5/145

(52) U.S. Cl. 600/365

(57) ABSTRACT

A device for the non-invasive measurement of a glucose level, body hydration or another characterizing parameter of body tissue comprises at least two coplanar waveguides arranged on a common support. An AC signal is applied to the first ends of the coplanar waveguides, and the signal arriving at the second end is measured. The coplanar waveguides have differing gap widths, such that their electric fields have different reach into the body tissue. This allows obtain depth resolved information about the permittivities of individual tissue layers and to obtain more accurate results.

### TECHNICAL FIELD

The invention relates to a device and a method for determining at least one characterizing parameter of body tissue, in particular living body tissue, such as glucose level or water content, by means of the application of electrical fields.

### BACKGROUND ART

WO 02/069791 describes a device for measuring blood glucose in living tissue. It comprises an electrode arrangement with a ground electrode and a signal electrode. A signal source applies an electrical AC-signal of known voltage or current through a resistor to the electrodes, and a detector determines the voltage over or current through the electrodes. This voltage or current depends on the dielectric properties of the tissue, measured as an impedance or admittance which, as it has been found, is indicative of the glucose level within the tissue.

WO 2005/120332 describes another embodiment of such a device where a plurality of electrical fields are generated by applying voltages to different configurations of the electrode arrangement, thereby generating fields of different spatial configurations within the tissue. This allows, for example, a reduction of the influence of surface effects on the measured signal.

These techniques allow to measure a characterizing parameter of living tissue, in particular the glucose level or water content, where this parameter affects the complex dielectric permittivity ∈(ω) of the tissue. They rely on applying an electrode arrangement to a skin region of the tissue and generating electrical fields within the tissue. For each field, a signal depending on the bulk dielectric properties as seen by the electrode arrangement is measured. The measured signal is then processed, e.g. using pre-recorded calibration data, in order to obtain the characterizing parameter, such as the glucose level.

### DISCLOSURE OF THE INVENTION

The object of the present invention is to provide a device and method of this type that further improves the accuracy of the measured characterizing parameter.

This object is achieved by the device and method according to the independent claims.

Accordingly, a device is provided that comprises several coplanar waveguides, with each waveguide having a center strip electrode between ground electrodes. At least some of the coplanar waveguides differ in their geometry in that they have different distances between their center strip electrode and their ground electrodes, such that, upon application of an electrical voltage between the center strip electrode and the ground electrodes, they generate electrical fields of different penetration.

The device further comprises a signal generator generating at least one AC signal, which is fed to a first end of said coplanar waveguides. A measuring unit is provided that measures N measured parameters mᵢ, with each measured parameter mᵢ being indicative of the signal emerging from the second end of each coplanar waveguide.

Finally, a control unit is provided that is adapted to determine the characterizing parameter P from said measured parameters mᵢ.

For example, the control unit may comprise a lookup table storing calibration coefficients that allow the conversion of said measured parameters mᵢ to said characterizing parameter P, with the calibration coefficients being recorded in calibration measurements.

The AC signal can be generated as an oscillating signal (such as a sine wave or a square wave), but it may also be generated by a single voltage pulse or a voltage step.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 is a sectional view of a coplanar waveguide,

FIG. 2 is a sectional view of a conductor-backed coplanar waveguide,

FIG. 3 shows a graphical representation of the measurement system based on the CPW,

FIG. 4 is a block diagram of a device for measuring a parameter,

FIG. 5 is a device carrying two CPWs as seen from the side facing the sample,

FIG. 6 is an alternative CPW geometry,

FIG. 7 shows a device with a two-layer skin region above it,

FIG. 8 shows a diagram of the dielectric constants and glucose levels as measured in a test measurement,

FIG. 9 shows the dermis permittivity (real part) and glucose levels during two test measurements, and

FIG. 10 shows the dermis permittivity (imaginary part) and glucose levels during the two test measurements.

### MODES FOR CARRYING OUT THE INVENTION

### 1. Introduction

### 1.1. Human Skin Structure

The skin can be basically divided into two major parts. The epidermis—the outer skin—comprises the stratum corneum, stratum granulosum and stratum spinosum, which forms a waterproof, protective covering over the human body surface. It does not contain any blood vessels and is nourished by diffusion from the dermis, the underlying skin layer. The underlying dermis is the layer of the skin that consists of connective tissue and cushions the body from stress and strain. The dermis is tightly connected to the epidermis by the basement membrane. It also harbors many nerve endings that provide the sense of touch It contains the hair follicles, sweat glands, sebaceous glands, apocrine glands and blood vessels. The blood vessels in the dermis provide nourishment and waste removal to and from its own cells as well as the stratum basale of the epidermis. A model of the human skin for electromagnetic simulations is described in detail in the next section.

### 1.1.1. Skin Structure and its EM Modelling

Table 1 summarizes, as an example, a dielectric model of the skin at the upper arm. The parameters of this model are given for static DC conditions only, which do not correspond to the dielectric behaviour in reality; but they provide a first estimate for an initial design. It has to be noted that the thickness of single layers strongly depends on the observation site of the human body.

TABLE 1
Dielectric model of human skin (static DC conditions)

| | | Rel. Permittivity, | Conductivity, |
|---|---|---|---|
| Layer | Thickness, t/m | εᵣ | σ/S/m |
| Sebum | 4E−6 | 25 | 1E−3 |
| Stratum | 15-100E−6 | 10 (higher | 1E−4 to 1E−5 |
| | | for humid) | |
| Epidermis | 100-200E−6 | 20 | 0.025 |
| Dermis | 1E−3 | 110 | 0.2 |
| Fat | 1.2E−3 | 20 | 2E−4 |
| Muscle | 20E−3 | 80 | 0.7 |

The sebum layer of the skin describes the substance secreted by the sebaceous glands. It is mainly consists of fat and the debris of dead fat-producing cells. Sebum protects and waterproofs hair and skin, and keeps them from becoming dry, brittle, and cracked. For electro-magnetic simulations, it is modelled to have a permittivity of some 25.

Strictly speaking, the stratum corneum is a part of the epidermis layer of the skin. It has, however, slightly different properties from the electro-magnetic point of view. Due to the different conductivity, it may be modeled as an additional layer. In physiological terms, stratum corneum is the outermost layer of the epidermis. It is mainly composed of dead cells. As these dead cells slough off, they are continuously replaced by new cells from the underlying layers. Cells of the stratum corneum contain keratin, a protein that helps keep the skin hydrated by preventing water evaporation. In addition, these cells can also absorb water, further aiding in hydration. The permittivity and conductivity of this layer is assumed to be variable and dependent on whether the skin is wet or not.

Due to the high concentration of protein fibres, the dermis layer has got a very high permittivity of ˜110, while the presence of the blood and the interstitial fluid increases its conductivity in comparison to the surrounding tissues.

The deeper layers, which have to be considered for the development of sensors having large electrode separations, are the fat and the muscle compartments. The fat is the main component of the subcutaneous tissue (also called hypodermis). The muscle tissue is set to be the boundary for the EM model as it is assumed to have relatively high values of thickness (20 mm) and conductivity (0.7 S/m).

### 1.2. Layer Model

In an advantageous embodiment of the present invention, it is assumed that a skin region to be tested is composed of a layer structure having a plurality of homogeneous layers i with i=1 . . . N and N>1, with layer N being the topmost layer, i.e. the layer comprising one or more of the outermost layers of the skin. The individual layers have thicknesses hᵢ. h₁ is assumed to be infinite. The other thicknesses h₂ . . . h_{N} may be equal or not equal to each other.

The linear response of each layer to an applied electric field is described by its permittivity ∈ᵢ. In general, the permittivity ∈ᵢ is a complex number having a real part ∈′ᵢ and an imaginary part ∈″ᵢ. In a simple model, the imaginary part ∈″ᵢ can be assumed to be zero (lossless case, zero conductivity), while a refined model can take non-zero imaginary parts ∈″ᵢ into account. Methods for calculating how an electrical field is affected by such multi-layer systems, and in particular what effective permittivity ∈_{eff} the field experiences, are known to the skilled person.

### 2. Sensor Implementations

### 2.1. Introduction

The present invention uses a sensor device that is able to perform a depth-resolved measurement on a skin region having a structure as described under section 1.2 in contact with the skin. This sensor comprises several coplanar waveguides as described below.

In general, such a sensor has N coplanar waveguides. The distances Wᵢ between the ground and signal electrodes of each coplanar waveguide differ from each other.

The sensor device is applied to the skin region under test with the electrodes of the coplanar waveguides being close to the topmost layer of the skin. The coplanar waveguides are then used to generate at least N electrical fields within the skin region, wherein the electrical fields have differing penetration depths into the said skin region. The different electrical fields can be applied sequentially, or (if a cross-talk between coplanar waveguides can be neglected or is compensated for) the fields can be applied concurrently.

The characteristics of the field distribution will be a function of the differing effective permittivities ∈_{ff}, depending on how far the fields reach into the skin/tissue. These effective permittivities describe the linear response (polarization) of the tissue to the fields.

For each field or coplanar waveguide, a “measured parameter” mᵢ is measured. This parameter may e.g. be the electrical impedance Z or capacitance C of the conesponding pair of electrodes, or a phase shift or damping coefficient for a signal passing through the coplanar waveguide, and it will depend on the effective bulk permittivity of the skin experienced by the coplanar waveguide.

Using e.g. the techniques as described below, the measured parameters mᵢ can be converted, by means of suitable calculations, into at least one “characterizing parameter” P, such as blood glucose concentration or a spatially resolved description of the water concentrations in the various tissue layers.

The present invention is based on the understanding that many characterizing parameters P of the tissue, such as blood glucose concentration, may strongly affect the permittivities ∈ᵢ of some layers and but have only weak (if any) influence on the permittivities of the other layers, while the permittivities of those other layers may be subject to other influences, such as environmental temperature or humidity, sweat, etc. Hence, a common analysis of the measured parameters mᵢ, which allows the derivation of spatially resolved information over the depth of the tissue, is able to provide a more accurate estimate for the characterizing parameter P.

### 2.2. Coplanar Waveguide Transmission Lines

### 2.2.1. Definition

The term “coplanar waveguide” (CPW) as used in this text and the claims is to be interpreted as an arrangement of an elongate center strip electrode between and at a distance from two ground electrodes. The signal electrode is much longer than it is wide. The signal and ground electrodes are mounted to the same surface of a non-conducting support. Optionally, a further ground electrode may be located on the opposite side of the support (an arrangement called “conductor-backed coplanar waveguide”, CBCPW). The electrodes may extend along a straight line, or they may be curved (e.g. in the form of a spiral) or polygonal (e.g. in the form of an L or a U).

Advantageously, the ground electrodes are much wider than the signal electrode as this design provides better field localization and is easier to model.

Furthermore, also advantageously, the width of the electrodes are constant along their longitudinal extension, and also the ground geometry does not change along the CPW, as this design is easiest to model. However, it may also be possible to vary these parameters along the CPW, e.g. by periodically changing the width of the signal electrode.

### 2.2.2. Examples

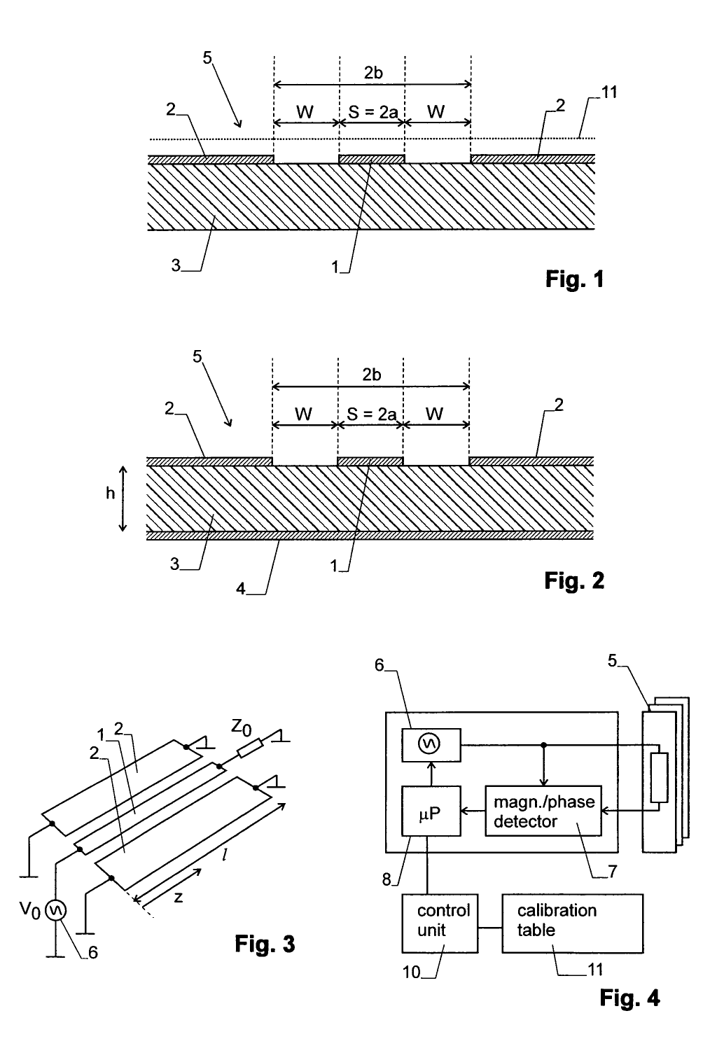

As shown in FIG. 1, an embodiment of a CPW on a dielectric substrate comprises a center strip electrode 1 conductor with (ideally) semi-infinite ground electrodes 2 on either side. Center strip electrode 1 and the ground electrodes 2 are arranged on a dielectric support 3. This structure supports a quasi-TEM mode of propagation. The coplanar waveguide 5 offers several advantages over a conventional microstrip line: First, it simplifies fabrication; second, it facilitates easy shunt as well as series surface mounting of active and passive devices; third, it eliminates the need for wraparound and via holes, and fourth, it reduces radiation loss. Furthermore the characteristic impedance is determined by the ratio of a/b, so size reduction is possible without limit, the only penalty being higher losses. In addition, a ground plane exists between any two adjacent lines; hence cross talk effects between adjacent lines are very weak.

The quasi-TEM mode of propagation on a CPW 5 has low dispersion and, hence, offers the potential to construct wide band circuits and components.

Coplanar waveguides can be broadly classified as follows:
Conventional CPW
Conductor backed CPW
Micromachined CPW

In a conventional CPW, the ground planes are of semi-infinite extent on either side. However, in a practical circuit the ground electrodes are made of finite extent. The conductor-backed CPW, as shown in FIG. 2, has an additional bottom ground electrode 4 at the surface of the substrate 3 opposite to electrodes 1 and 2. This bottom ground electrode not only provides mechanical support for the substrate but also acts as a heat sink for circuits with active devices. It also provides electrical shielding for any circuitry below support 3. A conductor backed CPW is advantageously used within this work.

As shown in dotted lines in FIG. 1, the electrodes 1, 2 may optionally be covered by a non-conductive cover layer 11 of known thickness and known dielectric properties. Such a cover layer can be used to avoid any possible electro-chemical effects at the electrodes, and it can also be used to change the effective penetration depths of the fields into the tissue.

### 2.3. Forward Problem for Conductor-Backed CPW (CBCPW)

In the following, the CBCPW 5 of FIG. 2 will be considered. The signal line has the width S and the gap width between signal and ground electrodes is W. The following annotations are used as well: S=2a and S+2W=2b.

First, the forward problem of the transmission line has to be solved, i.e. the calculation of the effective permittivity ∈_{eff} of the system depicted in FIG. 2. Usually, the shown configuration is used with air on top within the high-frequency systems (∈ᵣ₁=1). In measurement applications, the material under test (MUT) with permittivity ∈ₓ is placed on top of the transmission line (∈ᵣ₁=∈ₓ).

In order to be able to analytically state some simple relationships for the CPWs, a number assumptions and approximations have to be made. The main assumption is that the quasi-TEM (transversal electro-magnetic) wave propagation is dominant on the transmission line. This assumption implies that the losses in the metal strips and dielectric materials are low. This, of course, is not the case for human tissues. However, the analytic expressions allow to quickly analyze the sensor functionality before proceeding to the rigorous computer-aided full-wave analysis.

Based on this approximation, the analysis of Wen [1] can be expanded to the structure under consideration employing the procedure proposed by Gevorgian [2].

The effective permittivity as seen by the transmission line in FIG. 2 can be expressed by

∈_{eff}=1+q₁(∈ᵣ−1)+q₂(∈ₓ−1); (2.1)

with ∈ᵣ being the permittivity of support 3 and wherein

$$\begin{matrix}{q}_{1} = \frac{1}{1 + \frac{K ( {k}_{0} )}{K ( {k}_{0}^{′} )} \cdot \frac{K ( {k}^{′} )}{K ( k )}} ; & ( 2.2 ) \\ {q}_{2} = \frac{1}{1 + \frac{K ( {k}_{0}^{′} )}{K ( {k}_{0} )} \cdot \frac{K ( k )}{K ( {k}^{′} )}} . \\ = & ( 2.3 )\end{matrix}$$

The functions K(x) in Eqs. 2.2 and 2.3 are the complete elliptic integrals of the first kind. Re-arranging the Eqs.2.2 and 2.3, the effective permittivity of the system can be stated as:

$$\begin{matrix}{ɛ}_{eff} = {ɛ}_{r} \cdot {q}_{1} + {ɛ}_{x} \cdot {q}_{2} \\ = \frac{{ɛ}_{r}}{1 + \frac{K ( {k}_{0} )}{K ( {k}_{0}^{′} )} \cdot \frac{K ( {k}^{′} )}{K ( k )}} + \frac{{ɛ}_{x}}{1 + \frac{K ( {k}_{0}^{′} )}{K ( {k}_{0} )} \cdot \frac{K ( k )}{K ( {k}^{′} )}} ;\end{matrix} \qquad \begin{matrix}( 2.4 ) \\ ( 2.5 )\end{matrix}$$

The parameters kᵢ depend on the structure geometry and are defined as follows:

$$\begin{matrix}{k}_{0} = \frac{S}{S + 2 W} = \frac{a}{b} , & ( 2.6 ) \\ {k}_{0}^{′} = \sqrt{1 - {k}_{0}^{2}} ; \\ and & ( 2.7 ) \\ k = \frac{tanh ( \frac{π \cdot a}{2 h} )}{tanh ( \frac{π \cdot b}{2 h} )} , & ( 2.8 ) \\ {k}^{′} = \sqrt{1 - {k}^{2}} . & ( 2.9 )\end{matrix}$$

The characteristic impedance of the transmission line can then be calculated to:

$${Z}_{L} = \frac{60 π}{\sqrt{{ɛ}_{eff}}} \cdot {[ \frac{K ( k )}{K ( {k}^{′} )} + \frac{K ( {k}_{0} )}{K ( {k}_{0}^{′} )} ]}^{- 1} . \qquad ( 2.10 )$$

### 2.4. Permittivity Measurements Using CPW Lines

Due to several boundary conditions, such as size, form (planarity), bandwidth of operations, simplicity, non-invasiveness, the transmission-line technique is employed here. This technique is based on the fact that the wave propagation along the line is strongly affected by the permittivity of the dielectric material supporting the line. There are numerous publications which describe various aspects of the utilisation of this method for material characterisation from theoretical considerations of the inverse problem [4, 5] to practical sensor implementations [6-8].

Using Eq. (2.4), the inverse problem of the determination of the permittivity ∈ᵣ₁=∈ₓ can be solved using the following equation:

$${ɛ}_{x} = \frac{1}{{q}_{2}} ( {ɛ}_{eff} - {ɛ}_{r} \cdot {q}_{1} ) , \qquad ( 2.11 )$$

where q₁ and q₂ are defined by Eqs. (2.2) and (2.3), respectively.

### 2.4.1. Theory of the Sensor Operations

The unknown effective permittivity ∈_{eff} of the measurement system has to be determined experimentally. As described in the preface to this subsection, there are various methods to do so. FIG. 3 demonstrates graphically an advantageous method. A generator 6 provides a sinusoidal RF signal, which is applied to the input of center strip electrode 1. The voltage V(l) at the output of the center strip electrode 1 is measured. The propagating wave is attenuated and its velocity is reduced due to the higher permittivity of the medium in comparison to the free space. The following equation describes the voltage variation along the transmission line:

V(z)=Vₚ(z)·e^{-γ·z}+Vᵣ(z)·e^{γ·z}, (2.12)

where Vₚ(z) and Vᵣ(z) are the amplitudes of the signals propagating forth and back along the line. In case of the line termination with the specific impedance (usually 50Ω), the amplitude Vᵣ(z) of the reflected wave vanishes. Then, the voltage at the termination can be stated as

V(l)=V₀·e^{-γ·l}. (2.13)

The transfer function of the transmission line is then

H=e^{-γ·l}·e^{j·ω·t}= (2.14)

=e^{-α·l}·e^{j·(ω·t-β·l)} (2.15)

Comparing the transfer function with the forward transmission coefficient S₂₁=|S₂₁|·e^{-j·φ}, the following relationships for the attenuation and the phase of the measured signal at the CPW output can be defined:

$$\begin{matrix}α = - \frac{\vert {S}_{21} \vert}{l \cdot 20 log e} , & ( 2.16 ) \\ ϕ = 360 ° \cdot l \cdot f \sqrt{{μ}_{0} {ɛ}_{0}} \cdot \sqrt{{ɛ}_{eff}} . & ( 2.17 )\end{matrix}$$

It has to be noted at this point that the measured phase delay φₘ is usually higher than the value calculated in Eq. (2.17) due to the non-ideal matching of the measurement transmission line.

Combining Eqs. (2.11 and (2.17), the unknown permittivity ∈ₓ of the material under test can be defined as

$${ɛ}_{x} = \frac{1}{{q}_{2}} ( {[ \frac{{ϕ}_{0} - {ϕ}_{m}}{360 °} \cdot \frac{1}{l \cdot f \cdot \sqrt{{μ}_{0} {ɛ}_{0}}} ]}^{2} - {ɛ}_{r} \cdot {q}_{1} ) , \qquad ( 2.18 )$$

where φₘ is the measured phase delay by the sensor hardware in degrees, which differs from the phase delay over the transmission line. The base phase shift φ₀ is a constant defined by the sensor hardware. It has to be determined by a calibration procedure as described later.

### 2.4.2. Sensor Hardware

FIG. 4 shows the basic block diagram of the measurements system. A microwave signal is provided by an AC signal generator 6 and then applied to a first end (input end) of signal line 1 of coupling structure 5, which is brought in contact with the skin of a living human or non-human mammal. Coupling structure 5 is a CPW, in particular a CBCPW as described above, with the signal being applied as shown in FIG. 3. FIG. 4 schematically shows that there can be several such coupling structures.

The voltage at the second end (output end) of center strip electrode l of coupling structure 5 is fed to a magnitude/phase detector 7. In the present embodiment, this circuit compares the input and output signals of center strip electrode 1 and generates one or two DC signals, whose voltage is proportional to the magnitude ratio and/or phase difference between them. A microcontroller 8 digitizes and stores the measured data, which then can be used as the basis for calculations of the measure of interest. This sensor system is basically a simplified VNA (Vector Network Analyzer) on a board measuring the magnitude and phase of the forward transmission coefficient S₂₁. Detector 7 and microcontroller 8 together form a measuring unit for measuring the “measured parameter” mᵢ of each CPW.

Further, a control unit 10 is provided for processing the measured parameters mᵢ and for calculating the at least one characterizing parameter P, as defined above, therefrom. Control unit 10 may be implemented as part of microcontroller 8 or it may be a separate unit, such as an external computer.

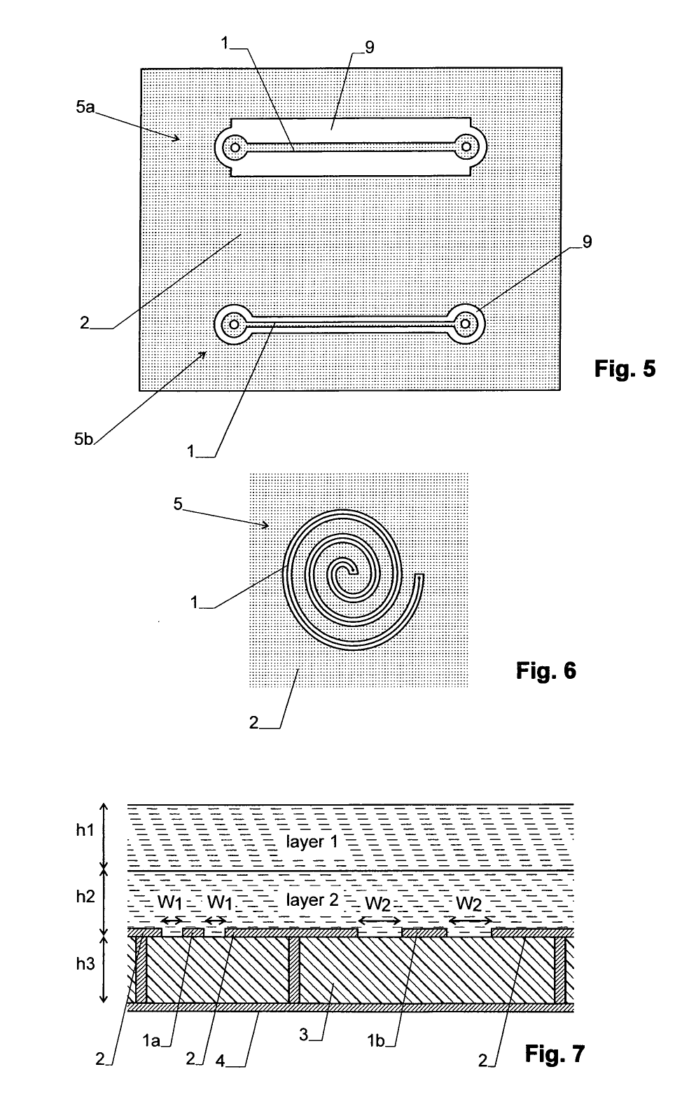

When several CPWs are part of the sensor device, a single signal generator 6 as shown in FIG. 5 can be used for feeding a common signal to all of them such that all CPWs are in operation at the same time. Alternatively, signal generator 6 may be adapted to subsequently feed a signal to each one of the CPWs such that the CPWs are operated in sequence, thereby minimizing crosstalk. Similarly, a measuring unit with several magnitude/phase detectors 7 may be provided, i.e. one detector 7 for each CPW, or a single magnitude/phase detector 7 can be switched between the output ends of the CPWs to sequentially measure the signals from all of them.

### 2.5. Electrode Geometries

It has been mentioned that the device is not limited to using straight CPWs. Nor can it use, for obvious reasons, infinitely long CPWs. FIG. 5 shows the design of an advantageous device with two CPWs of different geometry on a single support. In this figure, shaded areas denote the areas covered by center strip electrode 1 and the ground electrodes 2.

The device of FIG. 5 carries two CPWs 5a, 5b that have different gap widths W and therefore generate electrical fields having different penetration within the sample to be measured. CPW 5a has larger gap width W than CPW 5b.

As can be seen, the ground electrodes 2 are formed by a single, structured metal electrode, with each center strip electrode 1 being arranged in an opening 9 of said metal electrode.

As mentioned, the CPW does not necessarily have to extend along a straight line, but may also be curved. An example of a CPW having the form of a spiral is shown in FIG. 6.

In general, though, the cross section of the CPW (as shown in FIGS. 1 and 2) should be invariant along the extension z of the center strip electrode, such that the impedance Z does not vary along extension z. Otherwise, more complex models are required for the system modelling.

### 3. Inverse Problem for CBCPW

This section describes the detailed procedure derived to calculate the unknown value of the MUT (=Material Under Test) permittivity. First, a calibration procedure will be described. This procedure was designed to calculate the unknown parameters of the measurement system or parameters that were intentionally considered to be unknown. Then a mathematic description is defined, which is aimed at calculating the unknown permittivity of the MUT. Finally, a two-layer system is investigated. Using some approximations, both unknown permittivity values are calculated from measured results (“inverse profiling”).

Some assumptions have to be made in order to be able to analytically describe the measurements of the permittivities employing the proposed sensor structure.
Quasi-TEM wave propagation as described in Sec. 2.2
The capacitance values introduced by the radial signal junctions (i.e. the junctions at the ends of center strip electrode 1) can be accounted for by an additional length of the transmission lines. I.e., an ideal CPW with l_{eff}>l describes the behavior of the transmission line. This is a very valid assumption as the phase delay can be later easily be accounted for by the open coaxialcapacitance models.
In the case of two-layer MUT, the EM field induced by the transmission line with the shorter W=ΔGS distance is mostly confined within the first layer, i.e. permittivity variation within the second (deeper) layer does not affect the propagation properties of the transmission line. This condition can be assumed during the first stage of the mathematical considerations. The penetration depth is a very critical value as it depends on the material parameters, sensor geometry, and frequency of operations.

It must be noted that the above assumptions simplify an analytical analysis of the system. The invention, though, does not necessarily rely on them. If the assumptions are not met, the system can e.g. still be modeled numerically if no analytical description can be derived.

### 3.1. Calibration Procedure

In the following, an example of a calibration procedure for a geometry as shown in FIG. 2 (CBCPW) is described. The procedure was then tested on a device having copper electrodes, copper vias (lead throughs) and a Rogers RO4350b support material (∈ᵣ=3.66). The device had two CPWs having different widths W.

Eq. (2.18) is repeated below as (3.1). This relationship defines the unknown permittivity from the phase delay φₘ measured by the sensor system.

$${ɛ}_{x} = \frac{1}{{q}_{2}} ( {[ \frac{{ϕ}_{0} - {ϕ}_{m}}{360 °} \cdot \frac{1}{{l}_{eff} \cdot f \cdot \sqrt{{μ}_{0} {ɛ}_{0}}} ]}^{2} - {ɛ}_{r} \cdot {q}_{1} ) \qquad ( 3.1 )$$

In the above equation, q₁ and q₂ denote the so-called “filling factors” of the substrate and the unknown material, respectively. The value φ₀ is a constant base phase shift (for constant frequency and line dimensions) defined by the system, ƒ is the frequency of operation, μ₀ and ∈₀ are physical constants for absolute permeability and permittivity of the free space, respectively. Finally, l_{eff} is the effective length of the measurement transmission line. This length equals to the geometrical length l in the case of ideal CPW. In the current case of a real sensor system, the measured phase delay is slightly higher than it would be theoretically expected. This effect is assumed to be accounted for by an effective length l_{eff}>l as discussed above.

For fixed dimension and frequency, Eq. (3.1) can be rewritten in the following form

∈ₓ=C₁+C₂·(φ₀−φₘ)² (3.2)

The three unknown constants C₁, C₂, and φ₀ only depend on the sensor geometry and the operating frequency. They can be easily found if at least three measurements on materials with known permittivities (instead of the MUT) are performed. Assuming that the known calibration materials have permittivity values of ∈₁, ∈₂ and ∈₃, and the corresponding measured phase values are φ₁, φ₂ and φ₃ respectively, the calibration constants can be defined as follows:

$$\begin{matrix}{ϕ}_{0} = \frac{1}{2} \cdot \frac{( {ɛ}_{3} - {ɛ}_{2} ) \cdot {ϕ}_{1}^{2} - ( {ɛ}_{3} - {ɛ}_{1} ) \cdot {ϕ}_{2}^{2} + ( {ɛ}_{2} - {ɛ}_{1} ) \cdot {ϕ}_{3}^{2}}{( {ɛ}_{3} - {ɛ}_{2} ) \cdot {ϕ}_{1}^{ } - ( {ɛ}_{3} - {ɛ}_{1} ) \cdot {ϕ}_{2}^{ } + ( {ɛ}_{2} - {ɛ}_{1} ) \cdot {ϕ}_{3}^{ }} & ( 3.3 ) \\ {C}_{2} = \frac{{ɛ}_{2} - {ɛ}_{1}}{{( {ϕ}_{0} - {ϕ}_{2} )}^{2} - {( {ϕ}_{0} - {ϕ}_{1} )}^{2}} & ( 3.4 ) \\ {C}_{1} = {ɛ}_{1} + \frac{{ɛ}_{2} - {ɛ}_{1}}{1 - {( {ϕ}_{0} - {ϕ}_{2} / {ϕ}_{0} - {ϕ}_{1} )}^{2}} & ( 3.5 )\end{matrix}$$

The derived calibration procedure has to be performed only once for each single sensor. It has only to be repeated if the hardware (either electronics or the coupling structure) is changed. Using the found calibration constants, the permittivity of an unknown material can be calculated easily.

### Example

A sensor having two CPW transmission lines width gap widths 0.1 mm and 0.2 mm, respectively, and the length of 25 mm was calibrated at the frequency of 0.8 GHz using air (∈=1), ethanol (∈=16.34) and distilled water (∈=79.00). Using Eqs. (3.3)-(3.5) above, the following results were obtained for the parameters φ₀, C₁, C₂:
CPW with W=0.1 mm: φ₀=158.3, C₁=−4.104, C₂=0.00355
CPW with W=0.2 mm: φ₀=156.3, C₁=−5.859, C₂=0.00266

### 3.2. “Inverse Profiling” for Two-Layer Problem

FIG. 7 demonstrates a configuration for determining the permittivity values of two layers 1 and 2. In order to tackle this problem, at least two measurements have to be performed. The ansatz in this work is to use at least two CPWs with different values of the ground-to-signal distance W. In the embodiment of FIG. 7, the first CPW has a center strip electrode 1a and the second one a center strip electrode 1b, with corresponding gap distances W1 and W2, respectively.

Furthermore, for simplicity, an additional condition should advantageously be fulfilled, which was already defined at the beginning of the section: the field induced by the transmission line with the shorter W=ΔGS distance (i.e. ‘short’) is confined within the layer 2, i.e. permittivity variation within the deeper layer 1 does not affect the propagation properties of the transmission line with smaller W. In the following subsections, a procedure is described that allows to calculate the desired unknowns.

3.2.1. Forward Problem of CBCPW with a Two-Layer MUT

First, the forward problem, i.e. the calculation of the effective relative permittivity ∈_{eff} of the described structure, is solved. This is performed employing the conformal-mapping technique defined by Veyres and Hanna [9] for finite CPW and modified by Bedair and Wolff [4] for multi-layer structures. The described considerations are only valid if the permittivity of the supporting material is lower than the unknown permittivities (which is the case for biological tissues).

The effective relative permittivity of the structure depicted in FIG. 7 can, analogously to Eq. (2.1), be stated as:

∈_{eff}=∈₁·q₁+∈₂·q₂+∈₃·q₃ (3.7)

Again, q₁, q₂, q₃ are the filling factors for the layers 1-3, respectively. The approach uses an exact expression for the characteristic impedance

$${Z}_{0}^{a} = \frac{1}{{c}_{0} \cdot {C}_{t}^{a}} \qquad ( 3.8 )$$

where c₀=2.9979·10⁸ m/s is the speed of light and Cₜ^{a} is capacitance per unit area if the air-filled capacitors are considered (∈₁=∈₂=∈₃=1. Then, the characteristic impedance of the considered transmission line can be stated as:

$${Z}_{0} = \frac{{Z}_{0}^{a}}{\sqrt{{ɛ}_{eff}}} \qquad ( 3.9 )$$

The air-filled capacitors can be defined as:

$${C}_{i}^{a} = 2 {ɛ}_{0} \frac{K ( {k}_{i} )}{K ( {k}_{i}^{′} )} \\ with \\ ( i = I , II , III ) \qquad ( 3.10 )$$

with K(kᵢ) and K(kᵢ′) as the complete elliptic integral if the first kind similar to the Eq. (2.2) and (2.7) and (2.9). In our particular case, the kᵢ can be defined as follows:

$$\begin{matrix}{k}_{I} = \frac{S}{S + 2 W} = a / b ; & ( 3.11 ) \\ {k}_{II} = \frac{sinh ( \frac{π \cdot a}{2 {h}_{2}} )}{sinh ( \frac{π \cdot b}{2 {h}_{2}} )} ; & ( 3.12 ) \\ {k}_{III} = \frac{tanh ( \frac{π \cdot a}{2 {h}_{3}} )}{tanh ( \frac{π \cdot b}{2 {h}_{3}} )} & ( 3.13 )\end{matrix}$$

The following values can be determined from the geometry and assumptions made by Veyres and Hanna [9]:

$$\begin{matrix}{C}_{t}^{a} = {C}_{I}^{a} + {C}_{III}^{a} & ( 3.14 ) \\ {q}_{3} = \frac{{C}_{III}^{a}}{{C}_{t}^{a}} & ( 3.15 ) \\ {q}_{2} = \frac{{C}_{II}^{a}}{{C}_{t}^{a}} & ( 3.16 ) \\ {q}_{1} = \frac{{C}_{I}^{a} - {C}_{II}^{a}}{{C}_{t}^{a}} & ( 3.17 )\end{matrix}$$

Using the above expressions, the forward problem depicted in FIG. 7 reduces to

$${ɛ}_{eff} = \frac{{C}_{I}^{a} - {C}_{II}^{a}}{{C}_{I}^{a} + {C}_{III}^{a}} \cdot {ɛ}_{1} + \frac{{C}_{II}^{a}}{{C}_{I}^{a} + {C}_{III}^{a}} \cdot {ɛ}_{2} + \frac{{C}_{III}^{a}}{{C}_{I}^{a} + {C}_{III}^{a}} \cdot {ɛ}_{3} \qquad ( 3.18 )$$

with Cᵢ^{a} defined by Eq. (3.10).

### 3.2.2. A Method for the Solution of the Inverse Problem

In the following a possible solution for the inverse problem is presented. It is based on several assumptions, which will be defined within the course of explanation. The coupling structure used consists of two conductor-backed coplanar waveguides as shown in FIG. 7. The described solution comprises the following steps.

### Calibration of Both Sensor Configurations

This has to be performed according to the procedure described in Sec. 3.1. The calibration materials can be, for example: Air (∈₁=1), ethanol (∈₂), and distilled water (∈₃). Using Eqs. (3.3)-(3.5), the following two sets of calibrations constants for each frequency value can be defined:
φ₀ₛ, C₂ₛ, C₁ₛ, for the ‘short’ CPW (W small) and
φ₀ₗ, C₂ₗ, C₁ₗ, for the ‘long’ CPW (W large)

### Permittivity of Layer 2

Under the above assumption that the field induced by the ‘short’ CPW (=CPW with smaller gap width W) is confined within the layer 2, the permittivity of this layer can be calculated to be

∈₂=C₁ₛ+C₂ₛ·(φ₀ₛ−φₘₛ)² (3.19)

with φₘₛ being the phase-delay value measured over the ‘short’ CPW applied to the unknown material (MUT).

### Effective Permittivity as “Seen” by the ‘Long’ CPW

The following step is the calculation of the effective permittivity as “seen” by the ‘long’ CPW (=CPW with larger gap width W). It is the dielectric characteristic of the hypothetical material mixture between layers 1 and 2 that defines the propagation properties of the transmission line with the wide ground-to-signal distance. The relative permittivity of this material mixture can be calculated by Eq. (3.20)

∈₁=C^{1l}+C₂ₗ·(φ₀ₗ−φₘₗ)², (3.20)

where φₘₗ is the phase-delay value ascertained by the sensor over the ‘long’ CPW applied to (MUT).

In order to be able to define the effective permittivity of the assumed material mixture, let's assume that the layers 1 and 2 are merged and describe a material layer with infinite thickness and relative permittivity ∈₁. For this new two-layer system with the single-layer MUT, the effective permittivity can be written as:

∈_{eff,1}=q₂ₗ₂·∈₁+q₃ₗ₂·∈₃ (3.21)

∈₁ is defined in (3.20), ∈₃ is the relative permittivity of the supporting substrate, and the filling parameters q₂ₗ₂ and q₃ₗ₂ can be calculated by Eqs. (3.16) and (3.15), respectively. The corresponding parameters for the determination of the elliptic integrals can be determined:

$$\begin{matrix}{k}_{2 l 2} = \frac{{a}_{l}}{{b}_{l}} ; & ( 3.22 ) \\ {k}_{3 / 2} = \frac{tanh ( \frac{π \cdot {a}_{l}}{2 {h}_{3}} )}{tanh ( \frac{π \cdot {b}_{l}}{2 {h}_{3}} )} ; & ( 3.23 ) \\ {k}_{i}^{′} = \sqrt{1 - {k}_{i}^{2}} , \\ i = 2 l 2 , 3 l 2 & ( 3.24 )\end{matrix}$$

aₗ and bₗ are the geometric parameters of the ‘long’ CPW.

### Inverse Profiling of a Two-Layer MUT

Now, let's consider the original measurement problem depicted in FIG. 7. The permittivity value ∈₁ can be calculated from Eq. (3.18):

$${ɛ}_{1} = \frac{1}{{q}_{1 l}} ( {ɛ}_{eff , l} - {q}_{2 l} \cdot {ɛ}_{2} - {q}_{3 l} \cdot {ɛ}_{3} ) \qquad ( 3.25 )$$

Considering the fact that q₃ₗ=q₃ₗ₂ and using Eq. (3.21), the expression (3.25) reduces to:

$${ɛ}_{1} = \frac{1}{{q}_{1 l}} ( {q}_{2 l 2} \cdot {ɛ}_{l} - {q}_{2 l} \cdot {ɛ}_{2} ) \qquad ( 3.26 )$$

According to Eqs. (3.10)-(3.13) and (3.16), q₂ₗ is defined as

$${q}_{2 l} = \frac{\frac{K ( {k}_{2 l} )}{K ( {k}_{2 l}^{′} )}}{\frac{K ( {k}_{1 l} )}{K ( {k}_{1 l}^{′} )} + \frac{K ( {k}_{3 l} )}{K ( {k}_{3 l}^{′} )}} \qquad ( 3.27 )$$

with parameters kᵢ and kᵢ′ obtained as follows:

$$\begin{matrix}{k}_{1 l} = \frac{{a}_{l}}{{b}_{l}} ; & ( 3.28 ) \\ {k}_{2 l} = \frac{sinh ( \frac{π \cdot {a}_{l}}{2 {h}_{2}} )}{sinh ( \frac{π \cdot {b}_{l}}{2 {h}_{2}} )} ; & ( 3.29 ) \\ {k}_{3 l} = {k}_{3 / 2} = \frac{tanh ( \frac{π \cdot {a}_{l}}{2 {h}_{3}} )}{tanh ( \frac{π \cdot {b}_{l}}{2 {h}_{3}} )} ; & ( 3.30 ) \\ {k}_{i}^{′} = \sqrt{1 - {k}_{i}^{2}} & ( 3.31 )\end{matrix}$$

At this point, it has to be mentioned that the value of h₂ is not known. It is only assumed here that this parameter describes the “penetration” depth of the EM-field induced by the ‘short’ transmission line. Generally, this value depends on the dimension of the transmission line, parameters of the unknown material, and frequency of operation.

### 3.2.3. Summary

The derived procedure allows to calculate the two unknown permittivity values for a two-layer material under tests employing the CPW sensor with two transmission lines with different ground-to-signal distance dimensions. The procedure comprises the following steps:

(a) Calibrate the device by carrying out test measurements with single layer systems of known substances, such as air, ethanol and distilled water. This provides the calibration constants φ₀ₛ, C₂ₛ, C₁ₛ, for the CPW with smaller gap width W and φ₀ₗ, C₂ₗ, C₁ₗ, for the CPW with larger gap width W. This calibration has to be performed just once for every hardware configuration.

(b) Apply the device to the surface of an unknown two-layer system. Calculate the dielectric constant ∈₂ of layer 2 using Eq. (3.19) and the dielectric constant ∈₁ of layer 1 using Eqs. (3.26) and (3.20).

### 4. Applications

### 4.1. Test Measurements

Apart from some quick functionality tests on homogeneous materials, the above technology was applied to collect measured data from clinical trials. The tested device comprised two CPWs having gap widths W₁=0.1 mm and W₂=4 mm, respectively. The trials lasted some 10 hours each, and the device was placed on the upper left arm by the elbow of the patients. No test visit was conducted on consecutive days.

The model used for calculation was a simple two-layer system: “epidermis” with thickness of 0.4 mm (defined by the electromagnetic field penetration) and permittivity ∈ₑₚᵢ=∈₂ and “dermis” with infinite thickness and permittivity of ∈_{d}=∈₁. The permittivities were assumed to be complex valued.

During a testing visit, the level of the blood glucose was modified using an oral carbohydrate load. The subjects were asked to ensure that the last nutrition uptake was at least 10 hours before they arrived at the investigational site. During the procedure, a standardized breakfast or commercially available nutrition drink is consumed, whereupon the blood glucose level started to rise, reaching a peak and then falling back to a lower level.

During the whole procedure, glucose was repetitively sampled using invasive, conventional means, and the phase shifts φ and losses over both CPWs were measured as “measured parameters”. The permittivities ∈₁ and ∈₂ were calculated, as “characterizing parameters”, from the measured parameters.

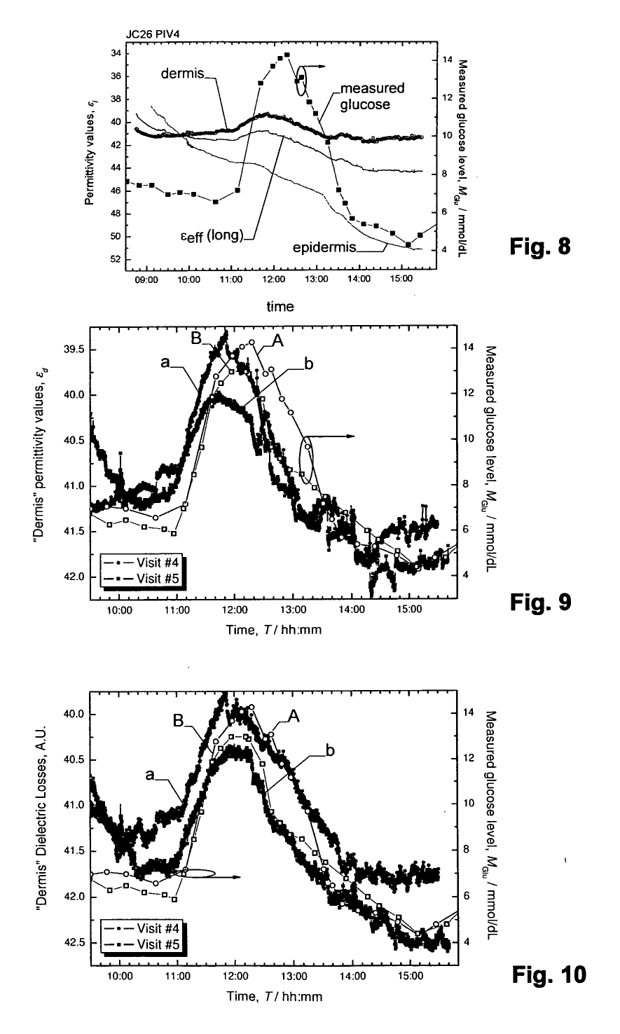

FIG. 8 shows the results of these measurements for a given trial run. As can be seen (from the curve “measured glucose”) the conventionally measured blood glucose was found to peak after food intake at 10:30 am. The effective permittivity ∈_{eff} (curve “∈_{eff} (long)”) as it was seen by the CPW of large gap (W=4 mm) as well as the calculated permittivities ∈₁ (curve “dermis”) and ∈₂ (curve “epidermis”) for both layers 1, 2 were also found to show some peaks that were temporally consistent with the glucose level change. However the effective permittivity ∈_{eff} and the permittivity ∈₂ of the top epidermis layer contained an underlying gradual decrease in trend that made a meaningful evaluation difficult. The permittivity ∈₁ of the dermis layer, however, showed a much more significant dependence on the glucose level change.

Hence, FIG. 8 clearly shows that, by using the signals measured by all CPWs and combining them to obtain a signal that is primarily dependent on properties of the “dermis” layer, a more accurate measure of the glucose level can be obtained.

This is further illustrated by FIGS. 9 and 10, which show the real part (FIG. 9) as well as the imaginary part (FIG. 10) of the dermis permittivity for two trial runs, together with reference blood glucose levels as obtained using conventional invasive measurements. Here, curves a and b show the permittivity values for the first and the second trial run, respectively, while curves A and B show the conventionally measured glucose levels for the same runs.

The measurements of FIGS. 8-10 were carried out at a frequency of 1.2 GHz.

### 4.2. Glucose Determination

As it is obvious from section 4.1, the glucose levels are strongly correlated with the “dermis” permittivity values, and can e.g. be calculated therefrom using simple calibration constants.

The methods for carrying out this type of calculation are known to the person skilled in the art. A detailed description can be found in WO 2005/053526. The disclosure of that document, in particular its section “Calibration”, is incorporated herein by reference. In particular, that document describes how to obtain a measure for the blood glucose level (or, in similar manner, some other characterizing parameter of the tissue) from a series of measured values sᵢ, using any suitable function F as defined in Eq. (1). In the context of the present invention, the measured values sᵢ can e.g. be the glucose levels ∈₁ and ∈₂ (or dermis glucose level ∈₁ only) as well as any further parameters that may effect the characterizing parameter, such as an environmental or surface temperature, as described in WO 2005/053526. Alternatively, instead of using the permittivities, the measured values sᵢ of WO 2005/053526 can comprise the measured parameters mᵢ (such as the phase shifts φ at the CPWs) of the present text, in particular if function F of WO 2005/053526 is designed to incorporate Eqs. (3.19), (3.26) and (3.20) above.

As it has been seen in section 4.1, the dermis permittivity provides a good indicator of blood glucose. For this reason, the electrical field of at least one of the CPWs should reach well into the dermis and the CPW i1 should therefore have a gap width W of at least 1 mm, in particular 1 to 4 mm. There should further be at least one CPW i2 with a gap width W of less than 1 mm, which allows to obtain a measure of the permittivity of the epidermis, which can be used to eliminate the epidermis permittivity from the signal obtained by the first CPW i1.

### 4.3. Determination of Other Characterizing Parameters

The present invention can also be used to determine one or more other characterizing parameters pᵢ, in addition to or alternatively to blood glucose concentration. One important parameter is skin hydration. Since water makes a major contribution to the permittivity value of the tissue, the knowledge of the permittivity values of the different layers of the tissue allows one to provide an estimate of water content for the given layers.

In a simple model based on Kraszewski mixture formula [10], it can be assumed that the volume fraction pᵢ of water in a material, tissue, or emulsion can be expressed as a function of measured permittivity ∈ᵢ and permittivities (real part of) of water (∈₁) and dry matter (∈₂):

$${p}_{i} = \frac{{ɛ}_{i}^{0.5} - {ɛ}_{2}^{0.5}}{{ɛ}_{1}^{0.5} - {ɛ}_{2}^{0.5}} \qquad ( 4.1 )$$

An additional capability of the described sensor and procedure is the determination of the water content (or content of another substance or material with known permittivity) in different layers not necessary lying on the surface. I.e. using the described system, it is possible to make depth profiling of the material under investigation assumed to be composed of two materials or two material groups.

### 4.4. Frequency

An important parameter of the measurements described here is the frequency of the applied fields. In general, CPW-type sensors operated in transmission, as described here, are especially suited for measurements in the range of approximately 50 MHz to 100 GHz. For too low frequencies, the necessary line length would become too long. The exact frequency to be used depends strongly on the characterizing parameter to be measured.

The device can also carry out measurements at more than one frequency, either concurrently or consecutively.

4.5. CPW Dimensions The primary factor determining the reach of the field of a CPW sensor into the body tissue is its gap width W. CPWs having a sufficiently large range of gap widths should be incorporated into the device for obtaining spatially resolved measurements of each skin layer having dielectric properties of interest.

In particular, at least one CPW should have a gap width W of 100 μm or less in order to obtain a measurement specific for the epidermis layer.

Similarly, at least one other CPW should have a gap width W of at least 1 mm in order to obtain a measurement indicative of dermis properties. In particular, the gap width of this CPW should be in a range of 1 to 4 mm since a CPW with a larger gap width will tend to create a field reaching into subdermal regions.

In some embodiments, e.g. for hydration measurements for evaluating the “total body water”, it may be of interest to reach even further into the body tissue, and in particular into subdermal regions, such as the muscle tissue. In that case, at least one of the CPWs should have a gap width W of at least 4 mm. It has been found that a dehydration of the body will first affect the water content in the muscle tissue, for which reason hydration measurements advantageously use CPWs with such large gap widths. In that case, a further CPW should be provided with a gap width W of less than 5 mm for eliminating the influence of any undesired parameter in the signal measured by the CPW of larger width.

### 4.6. Selective Layer Measurements

As mentioned in the examples for hydration and glucose measurements, some characterizing parameters P have a strong influence on the permittivity of at least a first layer of the tissue while the permittivity of at least a second layer above the first layer (i.e. closer to the surface than the first layer) is predominantly affected by other parameters. For example, blood glucose affects the permittivity of the dermis layer strongly, while the permittivity of the epidermis layer is predominantly affected by other factors, such as environmental temperature and humidity, general skin condition, etc. Similarly, total body water affects the permittivity of a subdermal layer while the permittivities of the dermis and epidermis layers are strongly affected by other factors.

The present technique is particularly suited for solving this type of problem by using the following steps:

1. Measuring the measured parameters mᵢ as described above, wherein
at least a first of the measured parameters mᵢ₁ of a layer i1 depends on the characterizing parameter P and a further parameter Q and
at least a second of the measured parameters mᵢ₂ of a layer i2 above layer i1 depends on the further parameter Q, but to a lesser degree on the characterizing parameter P.

(The term “further parameter” designates any parameter that significantly affects the permittivity of the second layer. The term “to a lesser degree” means that the parameter mᵢ₂ has a smaller dependence on characterizing parameter P or no such dependence at all.)

2. Combining the measured parameters mᵢ₁ and mᵢ₂ for obtaining a calculated value (such as the permittivity ∈ᵢ₁ of layer i1 using Eq. (3.19)) that depends less on the further parameter Q than measured parameter mᵢ₁.

3. Deriving the characterizing parameter P from the calculated value (as well as, where appropriate), any further parameters, such as temperature.

### 4.7. General Remarks

The number N of CPWs having different gap widths W depends on the application. For any depth-resolved measurement, N must be larger than 1. In the above examples, two CPWs were used, but the number N can easily be increased to higher values, such as 4 or more. In that case, the method for inverse profiling can be generalized to make a depth profile of a material having N layers. These layers can also be virtual and have only a theoretical depth. To perform a profiling for more than two layers, one can proceed as follows (1 is the most inner and N is the most top layer):
1. Consider the entire material consisting of two layers (consisting of several layers again). For example, to start profiling of a four-layer system using a measurement system with four CPWs, solve the two-layer problem for layers 4 and 3 using the measurements on the corresponding CPWs (e.g. 4 and 3).
2. In the next step, the parameters of the layer 2 can be calculated employing the measurements on the CPWs 3 and 2. In this case, the layers 4 and 3 are considered to be a single virtual layer 3*.
3. Proceed until all wanted parameters are calculated.

While there are shown and described presently preferred embodiments of the invention, it is to be distinctly understood that the invention is not limited thereto but may be otherwise variously embodied and practiced within the scope of the following claims.

### REFERENCES

[1] C. P. Wen, “Coplanar Waveguide: A Surface Strip Transmission Line Suitable for Nonreciprocal Gyromagnetic Device Applications,” in IEEE Trans Microwave Theory Techn., December 1969, pp. 1087-1090.
[2] S. Gevorgian, L. J. P. Linnér, E. L. Kollberg, “CAD Models for Shielded Multilayered CPW,” in IEEE Trans Microwave Theory Techn., April 1995, pp. 772-779.
[3] J. Baker-Jarvis, M. D. Janezic, B. F. Riddle, R. T. Johnk, P. Kabos, Ch. L. Holloway, R. G. Geyer, and Ch. A. Grosvenor, Measuring the Permittivity and Permeability of Lossy Materials: Solids, Liquids, Building Materials, and Negative-Index Materials. NIST Technical Note 1536, Boulder, Colo.: NIST, 2005.
[4] S. S. Bedair and I. Wolff, “Fast, Accurate and Simple Analytic Formulas for Calculating the Parameters of Supported Coplanar Waveguides for (M) MIC's,” in IEEE Trans. Microwave Theory Techn, vol. 40, January 1992, pp. 41-48.
[5] M. D. Janezic, D. F. Williams, “Permittivivty Characterization from TransmissionLine Measurements,” in IEEE MTT-S Int Microwave Symposium Dig., June 197, pp. 1343-1346.
[6] S. S. Stuchly and C. E. Bassey, “Microwave coplanar sensors for dielectric measurements,” in Meas Sci Technol., 1998, pp. 1324-1329.
[7] A. Raj, W. S. Holmes, and S. R. Judah, “Wide Bandwidth Measurement of Coinplex Permittivity of Liquids using Coplanar Lines,” in IEE Trans. Instr. Meas., vol. 50, August 2001.
[8] B. Kang, J. Cho, Ch. Cheon, Y. Kwon, “Nondestructive Measurements of Complex Permittivity and Permeability Using Multilayered Coplanar Waveguide Structures,” in IEEE Microwave Wireless Comp. Lett., vol. 15, May 2005
[9] C. Veyres and V. F. Hanna, “Extension of the application of conformal mapping techniques to coplanar lines with finite dimensions,” in Int. J. Electron., vol. 48, pp. 47-56, 1980.
[10] A. Kraszewski, S. Kulinski, and M. Matuszewski, “Dielectric properties and a model of biphase water suspension at 9.4 GHz,” Journal of Applied Physics 47, no. 4 (April, 1976): 1275-1277.

### REFERENCE FIGURES

1, 1a, 1b: center strip electrode
2: ground electrodes
3: support
4: bottom ground electrode
5, 5a, 5b: coplanar waveguide
6: signal generator
7: magnitude/phase detector
8: microcontroller
9: opening
10: control unit
11: cover layer
a: half width of signal line
b: half width of ground electrode distance
aₗ, bₗ: geometric parameters of “long” CPW
c₀: speed of light
C₁, C₂: device geometry constants, see Eq. 3.2
C_{I}, C_{II}, C_{III}: air-filled capacitances, see Eq. 3.10
Cₜ^{a}: capacitance per unit for air-filled capacitors, see Eq. 3.10
f: frequency
h, h3: height of support
h1, h2: height of layers of two-layer system (FIG. 7)
H: transfer function (eq. 2.14)
K(x): complete elliptic integral function
k₀, k′₀, k, k, k_{I}, k_{II}, k_{III}′: structural parameters, Eqs. 2.6ff, 3.11ff
l: length
l_{eff}: effective length, taking into account the effect of the signal junctions
mᵢ: measured parameter for layer i
N: number of layers
P, pᵢ: characterizing parameters
pᵢ₀ and pᵢ₁: calibration parameters, Eq. (4.1)
Q: non-characterizing parameter
q₁, q₂, q₃: filling factors, see Eq. (2.2), (2.3) and (3.7)
S: width of signal line
S₁₂: forward transmission coefficient
W, W₁, W₂, Wᵢ: width of gaps between signal line and ground, distance of electrode pairs
V(z), Vₚ(z), Vᵣ(z): voltages along the signal line (eq. 2.12)
z: position along center strip electrode
Z₀: characteristic impedance
Z_{L}: line impedance
ΔGS: =W, see above
∈₀: absolute permeability
∈₁, ∈₂ and ∈₃: permittivities of calibration media
∈_{eff}: effective permittivity
∈ᵣ: permittivity of support
∈ᵣ₁: permittivity of space above CPW
∈ₓ: unknown permittivity
φ: phase shift
φₘ: measured phase shift
φ₀: base phase shift
φ₁, φ₂ and φ₃: phase shift values measured for calibration media
γ: damping factor
μ₀: absolute permeability

1. A device for determining at least one characterizing parameter P of living body tissue, in particular a glucose level or water content, comprising
a number N>1 of coplanar waveguides, each coplanar waveguide comprising a center strip electrode between ground electrodes, wherein at least some of said coplanar waveguides have different gap widths between their center strip electrode and their ground electrodes for generating electrical fields of different reach,
a signal generator generating at least one AC signal, wherein first ends of said coplanar waveguides are connected to said signal generator,
a measuring unit, wherein a second ends of said coplanar waveguides are connected to said measuring unit for measuring N measured parameters mᵢ,
a control unit for determining said characterizing parameter P from at least part of said measured parameters mᵢ.

2. The device of claim 1, comprising at least one coplanar waveguide having a gap width of 100 μm or less.

3. The device of claim 1, comprising at least one co-planar waveguide having a gap width of at least 1 mm, in particular between 1 and 4 mm.

4. The device of claim 1, comprising at least one co-planar waveguide having a gap width of at least 4 mm.

5. The device of claim 1, wherein said signal generator generates an AC signal having a frequency of at least 50 MHz.

6. The device of claim 1, wherein said ground electrodes are wider than said center strip electrodes.

7. The device of claim 1, further comprising a non-conductive cover layer covering said electrodes.

8. Use of the device of claim 1, for measuring a glucose level.

9. Use of the device of claim 1, for measuring a water content.

10. A method for determining at least one characterizing parameter P of body tissue, in particular a glucose level or water content, comprising,
applying a number N>1 of coplanar waveguides to a skin region of said body tissue, each coplanar waveguide comprising a center strip electrode between ground electrodes, wherein at least some of said coplanar waveguides have different distances between their center strip electrode and their ground electrodes generating electrical fields of different reach by means of said coplanar waveguides by applying an AC signal to a first end of each coplanar waveguide,
measuring N measured parameters mᵢ depending on a signal exiting from a second end of each coplanar waveguide,
determining said characterizing parameter P from at least part of said measured parameters mᵢ.

11. The method of claim 10, wherein at least a first of the measured parameters mᵢ₁ of a layer i1 depends on the characterizing parameter P as well as on a further parameter Q and at least a second of the measured parameters mᵢ₂ of a layer i2 above said layer i1 depends on said further parameter Q and to a lesser degree on the characterizing parameter P, said method further comprising the step of
combining the measured parameters mᵢ1 and mᵢ2 for obtaining a calculated value (∈ᵢ1) that depends less on the further parameter Q than said measured parameter mᵢ1.

12. The method of claim 11, wherein said characterizing parameter P is a glucose level

13. The method of claim 12, wherein a coplanar waveguide i1 with a distance between its center strip electrode and its ground electrodes of at least 1 mm is used for measuring said measured parameter mᵢ1 and a coplanar waveguide i2 with a distance between its center strip electrode and its ground electrodes of less than 1 mm is used for measuring said measured parameter mᵢ2.

14. The method of claim 13, wherein said coplanar waveguide i1 has a distance between its center strip electrode and its ground electrodes of less than 4 mm.

15. The method of claim 11, wherein said characterizing parameter P is water content.

16. The method of claim 15, wherein a coplanar waveguide i1 with a distance between its center strip electrode and its ground electrodes of at least 4 mm is used for measuring said measured parameter mᵢ1 and a coplanar waveguide mᵢ2 with a distance between its center strip electrode and its ground electrodes of less than 5 mm is used for measuring said measured parameter mᵢ2.
